(19) United States
(12) Patent Application Publication
(10) Pub. No.: US 2025/0166029 A1
(43) Pub. Date: May 22, 2025

(54) METHOD AND DEVICE FOR PROVIDING PRICE INFORMATION FOR HEALTH DATA

(71) Applicant: SEOUL NATIONAL UNIVERSITY R&DB FOUNDATION, Seoul (KR)

(72) Inventor: Young Hee LEE, Seoul (KR)

(73) Assignee: SEOUL NATIONAL UNIVERSITY R&DB FOUNDATION, Seoul (KR)

(21) Appl. No.: 19/033,974

(22) Filed: Jan. 22, 2025

Related U.S. Application Data
(63) Continuation of application No. PCT/KR2024/007376, filed on May 30, 2024.

(30) Foreign Application Priority Data
May 30, 2023 (KR) 10-2023-0069033

(51) Int. Cl. G06Q 30/0283; G16H 10/20; G16H 10/60; G16H 50/30

(52) U.S. Cl. CPC G06Q 30/0283; G16H 10/20; G16H 10/60; G16H 50/30

(57) ABSTRACT

According to an embodiment, there are disclosed a device and a method for providing price information for health data, including: a process of acquiring sensitivity information indicating a degree of sensitivity to a plurality of pieces of health data and survey data from a plurality of surveys including asking price information for the plurality of pieces of health data; a process of acquiring individual correlation information that represents an individual correlation of asking price relative to sensitivity in each of the plurality of surveys based on the survey data; a process of acquiring a correlation trend indicator that represents an overall correlation of the asking price relative to sensitivity based on a regression result of the individual correlation information; and a process of providing price information determined for the plurality of pieces of health data based on the correlation trend indicator.

### CROSS-REFERENCE TO RELATED APPLICATION

Pursuant to 35 USC 120 and 365(c), this application is a continuation of International Application No. PCT/KR2024/007376 filed on May 30, 2024, and claims the benefit under 35 USC 119(a) of Korean Application No. 10-2023-0069033 filed on May 30, 2023, in the Korean Intellectual Property Office, the entire disclosure of which is incorporated herein by reference for all purposes.

### TECHNICAL FIELD

The technical field of the present disclosure relates to a method for providing price information for health data, and particularly, to a technology that provides price information based on a plurality of pieces of price information for a plurality of pieces of health data acquired from a plurality of survey participants.

### BACKGROUND

The Ministry of Employment and Labor and the Korea Occupational Safety and Health Agency have been promoting many projects to improve worker safety and health, but they are mainly focused on individual workers' health check-ups. In general, health check-ups for individuals are conducted primarily to determine the presence of diseases, and not for the purpose of providing personal health data to improve the quality of life, and, thus, it is difficult to collect personal health data. Therefore, surveys conducted online or offline have been used to collect health data from many people, and the acquired health data is being sold to companies or necessary organizations, such as the Korea Occupational Safety and Health Agency, as various kinds of health data.

However, there is a problem in that the criteria and pricing for selling health data acquired from a plurality of people through surveys are unclear, making it difficult to sell health data at more efficient prices. Currently, in South Korea, there is a lack of awareness regarding the collection and management of critical health data that is related to corporate productivity. As a result, there is a problem in that prices for health data cannot be appropriately determined based on prices corresponding to the criteria for selling health data. Therefore, countermeasures are needed to solve these problems.

### DISCLOSURE OF THE INVENTION

### Problems to Be Solved by the Invention

In view of the foregoing, the present disclosure is conceived to provide price information for health data by offering a service for providing price information determined for each of a plurality of pieces of health data based on asking price information for the plurality of pieces of health data acquired from a plurality of survey participants.

The problems to be solved by the present disclosure are not limited to the above-described problems. Although not described herein, other problems to be solved by the present disclosure can be clearly understood by a person with ordinary skill in the art from the following descriptions.

### Means for Solving the Problems

A first aspect of the present disclosure provides a method for providing price information for health data by a device, including: a process of acquiring sensitivity information indicating a degree of sensitivity to a plurality of pieces of health data and survey data from a plurality of surveys including asking price information for the plurality of pieces of health data; a process of acquiring individual correlation information that represents an individual correlation of asking price relative to sensitivity in each of the plurality of surveys based on the survey data; a process of acquiring a correlation trend indicator that represents an overall correlation of the asking price relative to sensitivity based on a regression result of the individual correlation information; and a process of providing price information determined for the plurality of pieces of health data based on the correlation trend indicator.

Also, the asking price information includes first asking price information corresponding to types of the plurality of pieces of health data and second asking price information corresponding to periods of time collecting the plurality of pieces of health data.

Further, the process of providing the determined price information includes: a process of providing type-specific price information corresponding to the types of the plurality of pieces of health data; and a process of providing period-specific price information corresponding to the periods of time collecting the plurality of pieces of health data, and the plurality of pieces of health data includes at least one of nutritional status, exercise status, skin condition, eating habits, personal characteristic information, health management information, and hereditary data.

Furthermore, in the process of acquiring the correlation trend indicator, the sensitivity is used as an independent variable and the asking price is used as a dependent variable to acquire the correlation trend indicator.

Moreover, the correlation trend indicator is determined by a linear regression function based on the regression result.

Also, the process of providing the determined price information includes: a process of acquiring actual purchasing frequency and purchase price information corresponding to each of the plurality of pieces of health data from an external server; and a process of updating the determined price information based on the actual purchasing frequency and purchase price information.

Further, the process of providing the determined price information includes: a process of acquiring survey data acquisition time for each of the plurality of surveys; a process of determining reliability of the survey data; and a process of updating the determined price information based on the survey data acquisition time and the reliability.

Furthermore, the process of acquiring the individual correlation information includes: a process of acquiring a plurality of pieces of additional health data that represents individual-specific characteristics; and a process of acquiring the individual correlation information based on whether there is a significant difference between the plurality of pieces of additional health data and the asking price relative to sensitivity.

Moreover, the plurality of pieces of additional health data includes at least one of the presence or absence of disease, gender, income, and age. The asking price relative to sensitivity is determined to have a positive relationship between the sensitivity information and the asking price information.

Also, the survey data acquisition time refers to when all the survey data for the plurality of surveys has been acquired, and the reliability of the survey data is determined based on the number of pieces of the survey data, participation rate, survey period, and survey response speed.

A second aspect of the present disclosure provides a device for providing price information for health data, including: a receiver that acquires sensitivity information indicating a degree of sensitivity to a plurality of pieces of health data and survey data from a plurality of surveys including asking price information for the plurality of pieces of health data; and a processor that acquires individual correlation information that represents an individual correlation of asking price relative to sensitivity in each of the plurality of surveys based on the survey data, acquires a correlation trend indicator that represents an overall correlation of the asking price relative to sensitivity based on a regression result of the individual correlation information, and provides price information determined for the plurality of pieces of health data based on the correlation trend indicator.

Also, the asking price information includes first asking price information corresponding to types of the plurality of pieces of health data and second asking price information corresponding to periods of time collecting the plurality of pieces of health data.

Further, the processor provides type-specific price information corresponding to the types of the plurality of pieces of health data and period-specific price information corresponding to the periods of time collecting the plurality of pieces of health data, and the plurality of pieces of health data includes at least one of nutritional status, exercise status, skin condition, eating habits, personal characteristic information, health management information, and hereditary data.

Furthermore, the processor acquires the correlation trend indicator by using the sensitivity as an independent variable and the asking price as a dependent variable.

A third aspect of the present disclosure provides a non-transitory computer-readable storage medium that stores a program to implement the method of the first aspect.

### Effects of the Invention

According to an embodiment of the present disclosure, sensitivity information and asking price information for a plurality of pieces of health data are acquired from a plurality of survey participants. Thus, it is possible to increase objectivity in determining an appropriate price.

Also, by considering an individual correlation of asking price relative to sensitivity in each of a plurality of surveys based on the plurality of pieces of health data and an overall correlation of the health data based on the individual correlation information, it is possible to determine a more accurate price and thus possible to improve efficiency.

Further, when price information is finally determined, asking price information is acquired for each type of the plurality of pieces of health data and for each period of time collecting the plurality of pieces of health data. Thus, it is possible to further improve efficiency in terms of accuracy.

Furthermore, a correlation trend indicator is acquired through regression analysis. Thus, it is possible to improve efficiency in analyzing the plurality of pieces of health data.

Moreover, it is possible to update the determined price information by further considering actual purchasing frequencies for the plurality of pieces of health data and also possible to improve efficiency by considering whether there is a significant difference based on a correlation among a plurality of factors.

The effects of the present disclosure are not limited to the above-described effects. Although not described herein, other effects of the present disclosure can be clearly understood by a person with ordinary skill in the art from the following descriptions.

### BRIEF DESCRIPTION OF THE DRAWINGS

FIG. 1 is a block diagram schematically illustrating a configuration of a device for providing price information for health data according to an embodiment.

FIG. 2 is a flowchart showing each operation process of the device for providing price information for health data according to an embodiment.

FIG. 3A and FIG. 3B show an example of acquiring asking price information corresponding to the type of health data according to an embodiment.

FIG. 4A and FIG. 4B show an example of acquiring asking price information corresponding to a period of time collecting health data according to an embodiment.

FIG. 5A and FIG. 5B are provided to explain a correlation trend indicator acquired according to an embodiment.

FIG. 6 shows an example of acquiring a plurality of pieces of additional health data according to an embodiment.

### BEST MODE FOR CARRYING OUT THE INVENTION

The advantages and characteristics of the present disclosure and a method of achieving the advantages and characteristics will be clear by referring to embodiments described below in detail together with the accompanying drawings. However, the present disclosure is not limited to embodiments disclosed herein but will be implemented in various forms. The embodiments are provided by way of example only so that a person with ordinary skill in the art can fully understand the disclosures of the present disclosure and the scope of the present disclosure.

The terms used herein are provided only for illustration of the embodiments but not intended to limit the present disclosure. As used herein, the singular terms include the plural reference unless the context clearly indicates otherwise. The terms “comprises” and/or “comprising” specify the presence of stated components, steps, operations, and/or elements, but do not preclude the presence or addition of one or more other components, steps, operations, and/or elements. Throughout the whole document, like reference numerals denote like parts, and the term “and/or” includes any and all combinations of one or more of the associated listed items. Although the terms “first”, “second”, and the like are used for describing various components, these components are not confined by these terms. These terms are merely used for distinguishing one component from the other components. Therefore, a first component to be mentioned below may be a second component in a technical concept of the present disclosure.

Unless otherwise defined, all terms (including technical and scientific terms) used herein have the same meaning as commonly understood by a person with ordinary skill in the art to which this invention belongs. It will be further understood that terms, such as those defined in commonly used dictionaries, should not be interpreted in an idealized or overly formal sense unless expressly so defined herein.

The spatially-relative terms such as “below”, “beneath”, “lower”, “above”, “upper”, etc. may be used herein for ease of description to describe the relationship of one element or component with another element(s) or component(s) as illustrated in the drawings. It will be understood that the spatially relative terms are intended to encompass different orientations of the element in use or operation, in addition to the orientation depicted in the drawings. For example, if the component in the drawings is turned over, components described as “below” or “beneath” other components would then be oriented “above” the other components. Thus, the exemplary term “below” can encompass both an orientation of above and below. The component may be otherwise oriented in another direction and the spatially-relative terms used herein interpreted accordingly.

Hereafter, various embodiments of the present disclosure will be described in detail with reference to the accompanying drawings.

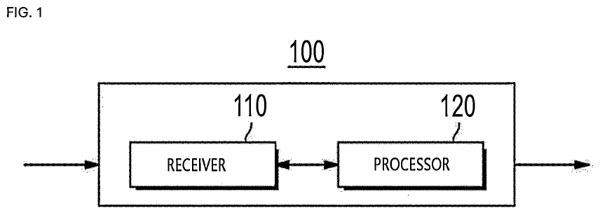

FIG. 1 is a block diagram schematically illustrating a configuration of a device 100 for providing price information for health data according to an embodiment.

Referring to FIG. 1, the device 100 may include a receiver 110 and a processor 120. In an embodiment, the receiver 110 may acquire sensitivity information indicating a degree of sensitivity to a plurality of pieces of health data and survey data from a plurality of surveys including asking price information for the plurality of pieces of health data through connection to the outside.

Further, it will be understood by a person with ordinary skill in the art that the device 100 for providing price information for health data may further include additional general-purpose components other than those shown in FIG. 1. For example, the device 100 for providing price information for health data may further include a memory (not shown) to store the survey data acquired by the receiver 110 from a plurality of servers, and individual correlation information and a correlation trend indicator acquired from the processor 120 as well as a display unit (not shown) to provide determined price information. It will be understood by a person with ordinary skill in the art that some of the components shown in FIG. 1 may be omitted according to another embodiment.

The device 100 for providing price information for health data according to an embodiment may be used by the user, and may be included in or linked to any type of handheld based wireless communication device having a touch screen panel, such as a mobile phone, a smartphone, a personal digital assistant (PDA), a portable multimedia player (PMP), or a tablet PC. In addition, the device 100 may include any devices on which an application can be installed and run, such as a desktop PC, a tablet PC, a laptop PC, and an IPTV including a set-top box.

The device 100 for providing price information for health data may be implemented as a device such as a computer operated through a computer program for performing functions described herein.

The device 100 for providing price information for health data according to an embodiment may include a system (not shown) to provide price information for health data and related servers (not shown), but is not limited thereto. The device 100 for providing price information for health data can support an application that offers a service for providing price information for health data according to an embodiment.

Hereafter, the device 100 for providing price information for health data according to an embodiment that independently provides a price determined for health data is mainly described, but the price may be provided by communication with a server as described above. That is, the device 100 for providing price information for health data according to an embodiment and the server may be integrally implemented in terms of function, and the server may be omitted. It can be understood that the present disclosure is not limited to any one embodiment. In other words, in an embodiment, the device 100 may be linked to the server, and a configuration for providing price information for health data as a price information providing device can be implemented by the server or may be implemented in the device 100. For example, the device 100 may operate as a server, and hereafter, it will be referred to uniformly as the device 100.

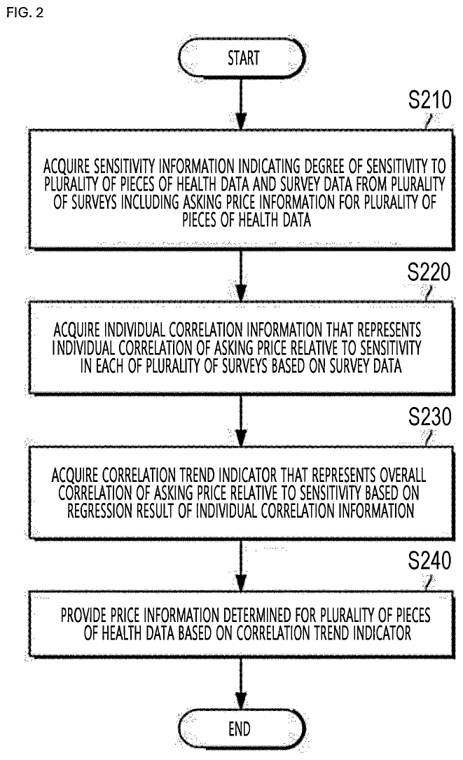

FIG. 2 is a flowchart showing each operation process of the device 100 for providing price information for health data according to an embodiment.

Referring to a process S210, the device 100 according to an embodiment may acquire sensitivity information indicating a degree of sensitivity to a plurality of pieces of health data and survey data from a plurality of surveys including asking price information for the plurality of pieces of health data. In an embodiment, the sensitivity information refers to a degree of sensitivity to each of a plurality of pieces of health data acquired from a plurality of people participating in online or offline surveys. Also, the sensitivity information refers to a degree of sensitivity that may affect determination of a selling price when each piece of the health data is sold or provided to third parties, such as companies or organizations. In an embodiment, the asking price information refers to a price for each of the plurality of pieces of health data which can be determined based on the acquired sensitivity information or an asking price corresponding to each of the plurality of pieces of health data which is acquired from the plurality of people participating in the surveys. In an embodiment, the asking price information may include first asking price information corresponding to types of the plurality of pieces of health data and second asking price information corresponding to periods of time collecting the plurality of pieces of health data. The first asking price refers to an asking price that is differently measured depending on the type of health data, and the second asking price refers to an asking price that is differently measured depending on whether a period of time collecting the plurality of pieces of health data is long or short. Therefore, the asking price information can be determined in consideration of both the type of health data and the period of time collecting health data.

Referring to a process S220, the device 100 according to an embodiment may acquire individual correlation information that represents an individual correlation of asking price relative to sensitivity in each of the plurality of surveys based on the survey data. In an embodiment, the individual correlation of asking price relative to sensitivity refers to a price corresponding to each level of sensitivity measured by the plurality of survey participants. For example, if the sensitivity levels are divided from 1 to 5, there may be a different asking price relative to sensitivity corresponding to each level based on the survey data of the plurality of survey participants. Further, the individual correlation information may be provided corresponding to each of the plurality of pieces of health data. For example, the asking price relative to sensitivity may vary among the plurality of people depending on the plurality of pieces of health data. Therefore, it is possible to acquire a plurality of asking prices relative to sensitivity for each of the plurality of pieces of health data through surveys and thus possible to acquire individual correlation information that represents an individual correlation for each of the plurality of pieces of health data.

The device 100 according to an embodiment may acquire a plurality of pieces of additional health data that represents individual-specific characteristics. Further, the device 100 may further acquire individual correlation information based on whether there is a significant difference between the plurality of pieces of additional health data and the asking price relative to sensitivity.

In an embodiment, the plurality of pieces of health data may include at least one of nutritional status, exercise status, skin condition, eating habits, personal characteristic information, health management information, and hereditary data which are general information related to health. For example, the nutritional status may include potassium levels, fatty acid concentration, calcium levels, etc., the exercise habits may include strength training, grip strength, aerobic fitness, ankle injury risk, etc., the skin condition may include freckles, acne, hair thickness, hair loss, etc., the personal characteristic information may include alcohol metabolism, alcohol dependence, insomnia, pain sensitivity, etc., the health management information may include motion sickness, obesity, body fat percentage, blood sugar, bone density, etc., and the hereditary data may include information about one's ancestors. Furthermore, the plurality of pieces of additional health data may include at least one of the presence or absence of disease, gender, income, and age which are individual-specific characteristics in addition to the general information related to health.

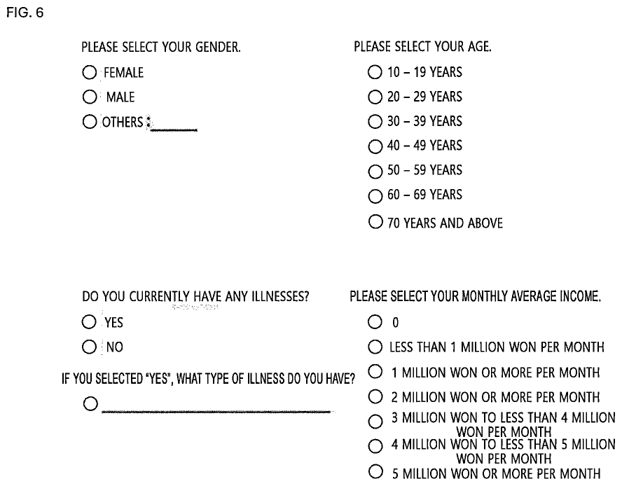

In an embodiment, the asking price relative to sensitivity may be determined to have a positive relationship between the sensitivity information and the asking price information. In an embodiment, the device 100 can acquire a more accurate correlation trend indicator by using the individual correlation information acquired when acquiring the plurality of pieces of additional health data as well as the individual correlation information acquired when acquiring the plurality of pieces of health data. This will be described with reference to FIG. 6.

Referring to FIG. 6, the device 100 may provide a plurality of options to answer questions corresponding to the plurality of pieces of additional health data. Therefore, the device 100 may further acquire individual correlation information based on the acquired plurality of pieces of additional health data. As described above, the device 100 may acquire the individual correlation information based on whether there is a significant difference between the plurality of pieces of additional health data and the asking price relative to sensitivity.

In an embodiment, the device 100 may first analyze a relationship between sensitivity information and asking price for health data (correlation analysis). In an embodiment, the device 100 may identify a significant difference in asking price based on sensitivity information since higher sensitivity is associated with a higher asking price. The device 100 may further acquire information on the intention to sell health data, which is obtained from an external server, depending on the presence or absence of disease. The device 100 may analyze a relationship between the intention to sell health data and the presence or absence of disease (cross-analysis).

In an embodiment, individuals with diseases are more likely to have a higher willingness to sell health data, the device 100 may determine that there is a statistically significant difference in the rate of intention to sell health data depending on the presence or absence of disease. Therefore, the device 100 can determine that there is a significant difference between the presence or absence of disease and an asking price relative to sensitivity. Also, the device 100 may analyze whether there is a difference between asking prices relative to sensitivity for health data based on survey data acquired by gender (independent sample t-test).

In an embodiment, asking prices relative to sensitivity are similar regardless of gender, and, thus, the device 100 may determine that gender does not cause a significant difference between asking prices relative to sensitivity. The device 100 may also analyze whether there is a difference between asking prices relative to sensitivity for health data based on survey data acquired by income or age (one-way analysis of variance).

In an embodiment, asking prices relative to sensitivity are similar regardless of income or age, and, thus, the device 100 may determine that income and age do not cause a significant difference between asking prices relative to sensitivity. Therefore, as described above, the device 100 may further acquire individual correlation information based on the presence or absence of a significant difference in each acquired factor.

Referring to a process S230, the device 100 according to an embodiment may acquire a correlation trend indicator that represents an overall correlation of the asking price relative to sensitivity based on a regression result of the individual correlation information.

In an embodiment, the correlation trend indicator refers to an indicator acquired as a result of regression of a plurality of pieces of individual correlation information, each representing an individual correlation of each of a plurality of pieces of health data. In an embodiment, when the correlation trend indicator is acquired through regression analysis, the sensitivity may be used as an independent variable and the asking price may be used as a dependent variable. The correlation trend indicator may be determined by a linear regression function based on the regression result since the effect of continuous sensitivity information on continuous asking price information is verified (increased sensitivity leads to a higher asking price). The regression refers to a method of analysis used to predict a continuous outcome variable by showing a relationship between two or more variables, and it may be used to identify changes in a dependent variable based on changes in at least one independent variable.

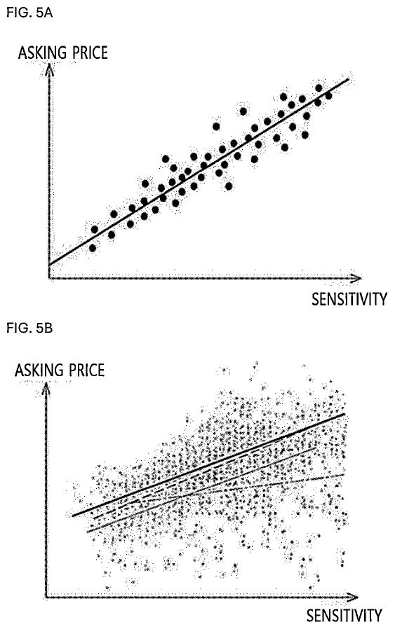

In an embodiment, an equation corresponding to the linear regression function, where the sensitivity is the independent variable and the asking price is the dependent variable, may be acquired through regression analysis. This will be described with reference to FIG. 5. Referring to FIG. 5, the independent variable is “sensitivity” mapped to an X-axis and the dependent variable is “asking price” mapped to a Y-axis. As shown in FIG. 5, a plurality of asking prices relative to sensitivity can be displayed as a plurality of points, and by performing regression, and a correlation trend indicator, which is a linear regression function, can be acquired through regression of the plurality of points.

In an embodiment, there may be a plurality of linear regression functions each corresponding to individual correlation information that represents an individual correlation of asking price relative to sensitivity for each of the plurality of pieces of health data. As shown in FIG. 5A, a plurality of asking prices relative to sensitivity corresponding to the plurality of pieces of health data (nutritional status, exercise status, eating habits, etc.) can be displayed as a plurality of points. As regression is performed, corresponding correlation trend indicators can be acquired. Thus, as shown in FIG. 5B, a plurality of different linear regression functions (asking price=a*sensitivity+b) corresponding to each of the plurality of pieces of health data can be created and acquired.

Referring to a process S240, the device 100 according to an embodiment may provide type-specific price information corresponding to types of the plurality of pieces of health data. Also, the device 100 may provide period-specific price information corresponding to periods of time collecting the plurality of pieces of health data.

As described in the process S210, the device 100 can acquire first asking price information corresponding to types of the plurality of pieces of health data and second asking price information corresponding to periods of time collecting the plurality of pieces of health data. Accordingly, the device 100 can provide type-specific price information corresponding to the first asking price information and period-specific price information corresponding to the second asking price information.

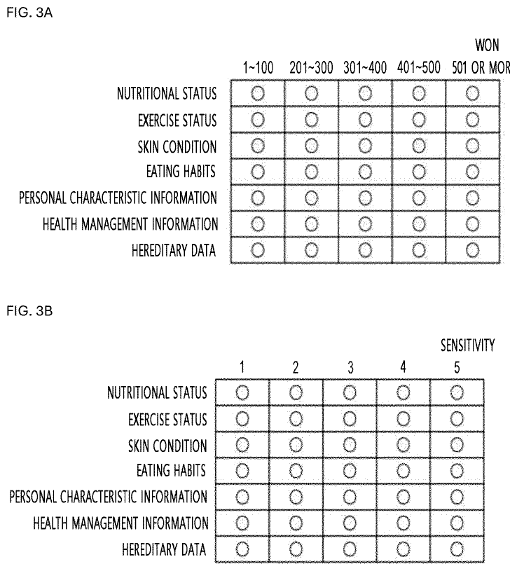

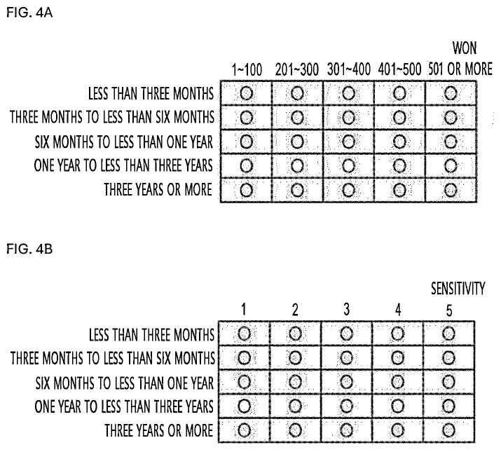

This can be explained with reference to FIG. 3A, FIG. 3B and FIG. 4. Referring to FIG. 3A and FIG. 3B, the device 100 may provide a plurality of options of desired selling prices and sensitivities for various types of health data.

In an embodiment, as shown in FIG. 3A and FIG. 3B, the various types of health data may include at least one of nutritional status, exercise status, skin condition, eating habits, personal characteristic information, health management information, and hereditary data. Therefore, the device 100 can acquire, from a plurality of survey participants, sensitivity information and asking price information corresponding to each of nutritional status, exercise status, skin condition, eating habits, personal characteristic information, health management information, and hereditary data.

Furthermore, referring to FIG. 4A and FIG. 4B, the device 100 may provide a plurality of options of desired selling prices and sensitivities for a plurality of periods of time collecting health data. In an embodiment, as shown in FIG. 4A and FIG. 4B, the plurality of periods of time collecting health data may include at least one of less than three months, three months to less than six months, six months to less than one year, one year to less than three years, and three years or more. Therefore, the device 100 may acquire, from a plurality of survey participants, sensitivity information and asking price information corresponding to each of less than three months, three months to less than six months, six months to less than one year, one year to less than three years, and three years or more.

In an embodiment, it will be understood by a person with ordinary skill in the art that types of health data, periods of time collecting health data, prices, and sensitivity levels are not limited to the above-described ones, but can be flexibly modified depending on circumstances or by the manager. Therefore, the device 100 may acquire survey data including sensitivity information and asking price information, acquire individual correlation information based on the survey data, and acquire a correlation trend indicator through regression of individual correlation information to provide determined price information.

The device 100 may according to an embodiment may acquire actual purchasing frequency and purchase price information corresponding to each of the plurality of pieces of health data from an external server. Also, the device 100 may update the determined price information based on the actual purchasing frequency and purchase price information.

In an embodiment, the external server refers to a server that can acquire information about the current status of the sale of health data, both online and offline, outside the service of the present disclosure. The device 100 may acquire purchasing frequency that represents how often the plurality of pieces of health data is actually sold, and purchase price information that represents actual selling prices. Based on the acquired actual purchasing frequency and price information, the device 100 may update the determined price information through regression. For example, a piece of health data with a higher actual purchasing frequency among the plurality of pieces of health data may be updated to have a higher price by a predetermined percent (e.g., 10 percent) than the other pieces of health data, and if actual purchase price information is higher than determined price information, the price may be updated by averaging the actual purchase price information and the determined price information.

In a converse case where actual purchasing frequency and price information are low, the determined price information may be updated in the opposite manner to the above-described method. In an embodiment, the determined price information may have objectivity depending on the linear regression function. However, it may be challenging to consider real-time situations. Therefore, the device 100 my update the determined price information by using actual purchasing frequency and purchase price information changed in real time depending on the sale of the plurality of pieces of health data acquired through the external server, and provide more appropriate determined price information.

Further, the device 100 according to an embodiment may acquire survey data acquisition time for each of the plurality of surveys, and determine reliability of the survey data. Accordingly, the device 100 may also update the determined price information based on the survey data acquisition time and the reliability of the survey data. In an embodiment, the survey data acquisition time refers to when all the survey data for the plurality of surveys has been acquired, and the reliability of the survey data is determined based on the number of pieces of the survey data, participation rate, survey period, and survey response speed.

In an embodiment, the survey data acquisition time corresponds to when all the first asking price information and sensitivity information corresponding to types of the plurality of pieces of health data and the second asking price information and sensitivity information corresponding to periods of time collecting the plurality of pieces of health data have been completely acquired. Therefore, the survey data acquisition time may be a factor used to assess a recency of the survey data.

The reliability of the survey data may be an additional factor to be considered after all the survey data is acquired, and can enhance accuracy. The number of pieces of the survey data for determining the reliability refers to the number of pieces of data included in the survey data, the survey period refers to a period of time conducting a survey, the participation rate refers to the number of survey participants relative to the survey period, and the survey response speed refers to the total time taken for each of pieces of the survey data when a plurality of survey participants responds to the survey.

The device 100 may assign a higher weight to survey data that has been acquired more recently as it is more likely to reflect current conditions. If the number of pieces of the survey data is too small, information may be insufficient, and if the number of pieces of the survey data is too large, a large amount of unnecessary data may be included. Therefore, the device 100 may assign a higher weight to survey data that corresponds to a predetermined number. Also, the accuracy of the survey data may decrease if the participation rate is too low. Therefore, the device 100 may assign a higher weight to survey data with a high participation rate.

Further, a longer survey period can increase the probability of including valid information, but an excessively long period may introduce unnecessary data. However, an excessively short survey period can increase the probability of missing valid information. Thus, the device 100 may assign a higher weight to survey data corresponding to a predetermined survey period. Further, an overly fast survey response speed may result in a lack of sincerity, while an overly slow survey response speed may indicate a lack of focus. Therefore, the device 100 may assign a higher weight to survey data acquired at a predetermined survey response speed corresponding to an average time relative to the number of pieces of the survey data. Accordingly, the device 100 may update the determined price information by assigning higher weights to more important survey data.

The device 100 according to another embodiment may assign different weights between a plurality of conditions, in addition to assigning a higher weight to a single condition for each factor. For example, the device 100 may update the determined price information based on weights assigned in descending order from survey data acquisition time to participation rate, survey period, the number of pieces of the survey data, and survey response speed. Herein, the survey data acquisition time is a factor that can confirm how recent survey data is, and serves as a factor for verifying accuracy. The other factors, such as participation rate, survey period, and survey response speed, are considered after survey data is completely acquired. Therefore, it is preferable to prioritize the survey data acquisition time, and, thus, the device 100 may assign the highest weight to the survey data acquisition time.

Furthermore, the participation rate refers to the number of survey participants relative to the survey period, and it can be important because a higher participation rate may result in a larger amount of survey data. A predetermined participation rate or higher can enhance reliability, and, thus, the device 100 may assign the second highest weight to the participation rate. Moreover, a longer survey period does not necessarily result in a higher accuracy, and, thus, the device 100 assigns a high weight to survey data corresponding to a predetermined survey period. However, due to the presence of additional considerations and the subjectivity associated with the predetermined survey period, accuracy may decrease slightly. Therefore, the survey period may be less important than the participation rate and thus may be assigned the third highest weight.

Also, the number of pieces of the survey data may be unnecessary when the survey data does not include important information, even if the number of pieces of the survey data reaches a predetermined number. When the survey data includes all the above-described health data, the number of pieces of the survey data may not make much of a difference. Therefore, the number of pieces of the survey data may be less important than the above-described three factors and thus may be assigned the fourth-highest weight. Further, the survey response speed can be important because it may indicate sincerity and focus. However, the survey response speed may vary depending on age, such as between people in their 20s and 70s, and thus may be assigned the fifth-highest weight. Thus, the device 100 can provide more accurate price information by updating the determined price information based on different weights assigned to the respective factors in a predetermined order.

FIG. 3A and FIG. 3B show an example of acquiring asking price information corresponding to the type of health data according to an embodiment. Referring to FIG. 3A and FIG. 3B, the device 100 may provide a plurality of options of desired selling prices and sensitivities for various types of health data.

In an embodiment, as shown in FIG. 3A and FIG. 3B, the various types of health data may include at least one of nutritional status, exercise status, skin condition, eating habits, personal characteristic information, health management information, and hereditary data. Therefore, the device 100 can acquire, from a plurality of survey participants, sensitivity information and asking price information corresponding to each of nutritional status, exercise status, skin condition, eating habits, personal characteristic information, health management information, and hereditary data. For example, as shown in FIG. 3A, the plurality of survey participants can select their asking price corresponding to each type of health data, and as shown in FIG. 3B, they can select sensitivity corresponding to each type of health data. In FIG. 3A, the optional asking prices are shown in the ranges of from 1 won to 100 won, from 201 won to 300 won, from 301 won to 400 won, from 401 won to 500 won, and 501 won or more, but are not limited thereto and can be replaced by other values. In FIG. 3B, the optional sensitivity levels are shown as 1, 2, 3, 4, and 5, but are not limited thereto and can be replaced by other values.

FIG. 4A and FIG. 4B show an example of acquiring asking price information corresponding to a period of time collecting health data according to an embodiment. Referring to FIG. 4A and FIG. 4B, the device 100 may provide a plurality of options of desired selling prices and sensitivities for a plurality of periods of time collecting health data.

In an embodiment, as shown in FIG. 4A and FIG. 4B, the plurality of periods of time collecting health data may include at least one of less than three months, three months to less than six months, six months to less than one year, one year to less than three years, and three years or more. Therefore, the device 100 may acquire, from the plurality of survey participants, sensitivity information and asking price information corresponding to each of less than three months, three months to less than six months, six months to less than one year, one year to less than three years, and three years or more. For example, as shown in FIG. 4A, the plurality of survey participants can select their asking price corresponding to each of the plurality of periods of time collecting health data, and as shown in FIG. 4B, they can select sensitivity corresponding to each of the plurality of periods of time collecting health data. In FIG. 4A and FIG. 4B, the plurality of periods of time collecting health data is shown as in the ranges of less than three months, three months to less than six months, six months to less than one year, one year to less than three years, and three years or more, but is not limited thereto and can be replaced by other values.

FIG. 5A and FIG. 5B are provided to explain a correlation trend indicator acquired according to an embodiment. Referring to FIG. 5A and FIG. 5B, as described above with reference to the process S230, the correlation trend indicator may be determined by a linear regression function based on a regression result since the effect of continuous sensitivity information on continuous asking price information is verified (increased sensitivity leads to a higher asking price). Referring to FIG. 5A and FIG. 5B, a plurality of asking prices relative to sensitivity can be displayed as a plurality of points, and by performing regression, and a correlation trend indicator, which is a linear regression function, can be acquired through regression of the plurality of points.

In an embodiment, there may be a plurality of linear regression functions each corresponding to individual correlation information that represents an individual correlation of asking price relative to sensitivity for each of the plurality of pieces of health data. As shown in FIG. 5A, a plurality of asking prices relative to sensitivity corresponding to the plurality of pieces of health data (nutritional status, exercise status, eating habits, etc.) can be displayed as a plurality of points. As regression is performed, corresponding correlation trend indicators can be acquired. Thus, as shown in FIG. 5B, a plurality of different linear regression functions (asking price=a*sensitivity+b) corresponding to each of the plurality of pieces of health data can be created and acquired.

FIG. 6 shows an example of acquiring a plurality of pieces of additional health data according to an embodiment. Referring to FIG. 6, as described above with reference to the process S220, the device 100 can acquire a more accurate correlation trend indicator by using the individual correlation information acquired when acquiring the plurality of pieces of additional health data as well as the individual correlation information acquired when acquiring the plurality of pieces of health data. As shown in FIG. 6, the device 100 may provide a plurality of options to answer questions corresponding to the plurality of pieces of additional health data. Therefore, the device 100 may further acquire individual correlation information based on the acquired plurality of pieces of additional health data. As described above, the device 100 may acquire the individual correlation information based on whether there is a significant difference between the plurality of pieces of additional health data and the asking price relative to sensitivity. Based on the acquired individual correlation information, the device 100 may acquire a final correlation trend indicator.

According to an embodiment, by considering an individual correlation of asking price relative to sensitivity in each of a plurality of surveys based on the plurality of pieces of health data and an overall correlation of the health data based on the individual correlation information, it is possible to determine a more accurate price and thus possible to improve efficiency. Further, when price information is finally determined, asking price information is acquired for each type of the plurality of pieces of health data and for each period of time collecting the plurality of pieces of health data. Thus, it is possible to further improve efficiency in terms of accuracy. Furthermore, a correlation trend indicator is acquired through regression analysis. Thus, it is possible to improve efficiency in analyzing the plurality of pieces of health data. Moreover, it is possible to update the determined price information by further considering actual purchasing frequencies for the plurality of pieces of health data and also possible to improve efficiency by considering whether there is a significant difference based on a correlation among a plurality of factors.

Various embodiments as set forth herein may be implemented as software including one or more instructions that are stored in a storage medium (e.g., a memory) that is readable by a machine (e.g., a display device or a computer). For example, the processor 120 of the machine may invoke at least one of the one or more instructions stored in the storage medium, and execute it. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Herein, the term “non-transitory” simply means that the storage medium is a tangible device, and does not include a signal (e.g., an electromagnetic wave), but this term does not differentiate between where data are semi-permanently stored in the storage medium and where the data are temporarily stored in the storage medium.

According to an embodiment, the method according to various embodiments of the present disclosure may be included in a computer program product and provided. The computer program product may be transacted as a commodity between a seller and a buyer. The computer program product may be distributed in the form of a device-readable storage medium (e.g., compact disc read only memory (CD-ROM)), or may be distributed (e.g., downloaded or uploaded) directly or on-line through an application store (e.g., Play Store™) or between two user devices (e.g., smart phones). In the case of on-line distribution, at least a portion of the computer program product may be at least temporarily stored or temporarily generated on a device-readable storage medium such as a server of a manufacturer, a server of an application store, or a memory of a relay server.

The present disclosure has been described with reference to the accompanying drawings, but is not limited to the embodiments and drawings. It can be understood by a person with ordinary skill in the art that the present disclosure is implemented as being modified and changed without departing from the spirit and the scope of the present disclosure. Accordingly, the above-described methods should be considered in descriptive sense only and not for purposes of limitation. Although explaining the embodiments of the present disclosure and explaining the operation and effect according to the constitution of the present disclosure have not been explicitly described, it is needless to say that a predictable effect is also recognized by the constitution. Also, the technical scope of the present disclosure is defined not by the detailed description of the invention but by the appended claims, and all differences within the scope will be construed as being comprised in the present disclosure.

We claim:

1. A method for providing price information for health data, comprising:
acquiring sensitivity information indicating a degree of sensitivity to a plurality of pieces of health data and survey data from a plurality of surveys including asking price information for the plurality of pieces of health data;
acquiring individual correlation information that represents an individual correlation of asking price relative to sensitivity in each of the plurality of surveys based on the survey data;
acquiring a correlation trend indicator that represents an overall correlation of the asking price relative to sensitivity based on a regression result of the individual correlation information; and
providing price information determined for the plurality of pieces of health data based on the correlation trend indicator.

2. The method for providing price information for health data of claim 1, wherein the asking price information includes:
first asking price information corresponding to types of the plurality of pieces of health data; and
second asking price information corresponding to periods of time collecting the plurality of pieces of health data.

3. The method for providing price information for health data of claim 1,
wherein the process of providing the determined price information includes:
providing type-specific price information corresponding to the types of the plurality of pieces of health data; and
providing period-specific price information corresponding to the periods of time collecting the plurality of pieces of health data,
wherein the plurality of pieces of health data includes at least one of nutritional status, exercise status, skin condition, eating habits, personal characteristic information, health management information, and hereditary data.

4. The method for providing price information for health data of claim 1,
wherein in the process of acquiring the correlation trend indicator, the sensitivity is used as an independent variable and the asking price is used as a dependent variable to acquire the correlation trend indicator.

5. The method for providing price information for health data of claim 4,
wherein the correlation trend indicator is determined by a linear regression function based on the regression result.

6. The method for providing price information for health data of claim 1,
wherein the process of providing the determined price information includes:
acquiring actual purchasing frequency and purchase price information corresponding to each of the plurality of pieces of health data from an external server; and
updating the determined price information based on the actual purchasing frequency and purchase price information.

7. The method for providing price information for health data of claim 5,
wherein the process of providing the determined price information includes:
acquiring survey data acquisition time for each of the plurality of surveys;
determining reliability of the survey data; and
updating the determined price information based on the survey data acquisition time and the reliability.

8. The method for providing price information for health data of claim 1,
wherein the process of acquiring the individual correlation information includes:
acquiring a plurality of pieces of additional health data that represents individual-specific characteristics; and
acquiring the individual correlation information based on the plurality of pieces of additional health data and whether there is a significant difference between the asking prices relative to sensitivity.

9. The method for providing price information for health data of claim 8,
wherein the plurality of pieces of additional health data includes at least one of the presence or absence of disease, gender, income, and age, and
the asking prices relative to sensitivity are determined to have a positive relationship between the sensitivity information and the asking price information.

10. The method for providing price information for health data of claim 7,
wherein the survey data acquisition time refers to when all the survey data for the plurality of surveys has been acquired, and
the reliability of the survey data is determined based on the number of pieces of the survey data, participation rate, survey period, and survey response speed.

11. A device for providing price information for health data, comprising:
a receiver that acquires sensitivity information indicating a degree of sensitivity to a plurality of pieces of health data and survey data from a plurality of surveys including asking price information for the plurality of pieces of health data; and
a processor that acquires individual correlation information that represents an individual correlation of asking price relative to sensitivity in each of the plurality of surveys based on the survey data, acquires a correlation trend indicator that represents an overall correlation of the asking price relative to sensitivity based on a regression result of the individual correlation information, and provides price information determined for the plurality of pieces of health data based on the correlation trend indicator.

12. The device for providing price information for health data of claim 11,
wherein the asking price information includes:
first asking price information corresponding to types of the plurality of pieces of health data; and
second asking price information corresponding to periods of time collecting the plurality of pieces of health data.

13. The device for providing price information for health data of claim 11,
wherein the processor provides type-specific price information corresponding to the types of the plurality of pieces of health data and period-specific price information corresponding to the periods of time collecting the plurality of pieces of health data, and
the plurality of pieces of health data includes at least one of nutritional status, exercise status, skin condition, eating habits, personal characteristic information, health management information, and hereditary data.

14. The device for providing price information for health data of claim 11,
wherein the processor acquires the correlation trend indicator by using the sensitivity as an independent variable and the asking price as a dependent variable.

15. A non-transitory computer-readable storage medium that stores a program to implement the method for providing price information for health data of claim 1.
